(19) United States
(12) Patent Application Publication
(10) Pub. No.: US 2003/0004385 A1
(43) Pub. Date: Jan. 2, 2003

(54) Process of treating an olefin isomerization catalyst and feedstock

(76) Inventors: Robert J. Gartside, Summit, NJ (US); Marvin I. Greene, Wayne, NJ (US)

Correspondence Address: Peter G. Dilworth, DILWORTH & BARRESE, LLP, 333 Earle Ovington Blvd., Uniondale, NY, 11553, US

(21) Appl. No.: 09/863,974

(22) Filed: May 23, 2001

(51) Int. Cl. C07C 5/23; C07C 5/27

(52) U.S. Cl. 585/664; 585/670; 585/671

(57) ABSTRACT

A process is provided for treating a basic metal oxide olefin isomerization catalyst, such as magnesium oxide. The catalyst is activated by contact with a deoxygenated nitrogen under activation conditions. The olefin isomerization process and catalyst described herein are advantageously used for the production of a terminal olefin such as 1-butene from an internal olefin such as 2-butene.

### BACKGROUND OF THE INVENTION

1. Field of the Invention

The present invention relates to a process for treating an olefin isomerization catalyst and the feedstock to the olefin isomerization process to improve the active life of the isomerization reaction system.

2. Description of the Related Art There is a growing need for terminal (alpha) olefins such as 1-butene or 1-hexene. The commercial production of alpha olefins is usually accomplished by the isolation of the alpha olefin from a hydrocarbon stream containing a relatively high concentration of the 1-isomer. For example, 1-butene can be isolated from the C₄ product of steam cracking. Steam cracking C₄ streams contain not only the 1-butene stream but also 2-butene, isobutylene, butadiene and both normal and iso butanes. The 1-butene is isolated by first separating butadiene by extractive distillation or removing butadiene by hydrogenation. Isobutylene can be removed either by reaction (e.g. reaction with methanol to form MTBE), or by fractionation, with the remaining n-butenes being separated by distillation into a 1-butene overhead stream and a 2-butene bottom product. An alternate production process for alpha olefins involves the dimerization of ethylene to form 1-butene or the trimerization of ethylene to form 1-hexene. Other methods include molecular sieve adsorption of the linear olefins (used for low concentrations).

Another process for providing alpha olefins is catalytic isomerization from internal olefins, which accomplishes the shifting of the double bond in an olefin molecule from, for example, an internal position (2-butene) to a terminal position (1-butene). High temperatures favor the isomerization of internal olefin to the alpha olefin. However, high temperature tends to cause catalyst coking which shortens catalyst life. The duration of catalyst activity is a significant factor with respect to the economic viability of a process. The more often a process has to be interrupted for catalyst regeneration the more costly the process becomes. Hence, a process for maintaining peak catalyst activity over a longer period of time at high temperature is a significant advantage for olefin isomerization.

### SUMMARY OF THE INVENTION

A process for activating a basic metal oxide isomerization catalyst is provided herein which comprises contacting the basic metal oxide catalyst under activation conditions with a dry inert gas containing not more than about 5 ppm molecular oxygen by volume.

Further provided is a process of treating the olefin isomerization feedstock by removing residual amounts of molecular oxygen therefrom.

The invention herein advantageously provides a basic oxide isomerization catalyst possessing an extended period of catalyst activity at relatively high isomerization temperatures. The isomerization process is advantageously used for the isomerization of internal olefins such as 2-butene to terminal olefins such as 1-butene.

### BRIEF DESCRIPTION OF THE DRAWINGS

Various embodiments of the invention are described herein with reference to the drawings wherein:

FIG. 1 is a schematic flow diagram of a process for treating a mixture of C₄ compounds from a cracker;

FIG. 2 is a schematic flow diagram of the olefin isomerization process of the present invention; and,

FIG. 3 is a schematic flow diagram of a catalyst regeneration system;

FIG. 4 is a chart illustrating the 1-butene olefin isomerization conversion over time for a catalyst treated in accordance with the process of the present invention; and,

FIG. 5 is a chart illustrating the 1-butene olefin isomerization conversion over time for a catalyst treated by conventional methods.

### DETAILED DESCRIPTION OF PREFERRED EMBODIMENT(S)

The olefin isomerization process herein is directed to the conversion of internally olefinic compounds to terminally olefinic compounds. While the process is described below particularly with reference to the conversion of 2-butene to 1-butene, the conversion of any internally olefinic compound to the terminally olefinic isomer is encompassed within the scope of the invention. Thus, for example, the conversion of 2-pentene to 1-pentene, 2-hexene or 3-hexene to 1-hexene, 2-heptene or 3-heptene to 1-heptene, and the like are also contemplated.

In a typical olefins plant, saturated hydrocarbons are converted to a mixture of olefins by a cracking process such as thermal cracking, steam cracking, fluid catalytic cracking and the like.

The resultant effluent from that cracking reaction is separated into carbon number fractions using a series of distillation columns and refrigerated heat exchange. In one sequence, a demethanizer is used for the removal of methane and hydrogen followed by a deethanizer for the removal of ethane, ethylene, and C₂ acetylene. The bottoms from this deethanizer tower consist of a mixture of compounds ranging in carbon number from C₃ to C₆. This mixture is separated into different carbon numbers, typically by fractionation.

The C₃ cut, primarily propylene, is removed as product and is ultimately used for the production of polypropylene or as a feedstock for synthesis of cumene or propylene oxide or acrylonitrile or other important chemical intermediates. The methyl acetylene and propadiene (MAPD) impurities must be removed either by fractionation or hydrogenation. Hydrogenation is preferred since some of these highly unsaturated C₃ compounds end up as propylene thereby increasing the yield.

The C₄ cut consisting of C₄ acetylenes, butadiene, iso and normal butenes, and iso and normal butane can be processed in many ways. A typical steam cracker C₄ cut contains components as set forth in Table 1. Table 1 is given for purposes of exemplification only. Component percentages of C₄ streams can be outside of the ranges given in Table 1.

TABLE 1

| | |
|---|---|
| C₄ acetylenes | trace |
| butadiene | 30-40 wt. percent |
| 1-butene | 10-20 wt. percent |
| 2-butene | 5-15 wt. percent |
| isobutene | 20-40 wt. percent |
| iso & normal butane | 5-15 wt. percent |

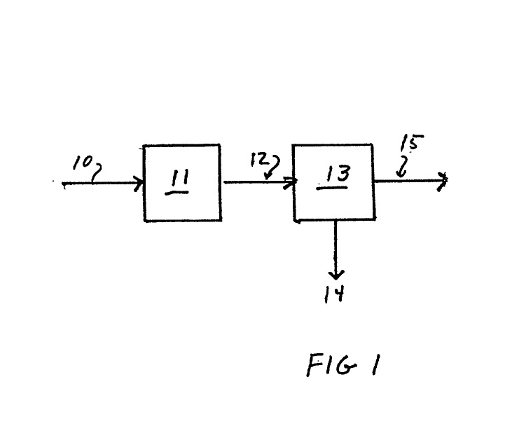

In a preferred method the processing of the C₄ stream is diagrammatically illustrated in FIG. 1. A stream 10 containing a mixture of C₄ components is sent to a catalytic distillation/hydrogenation unit 11 for hydrogenating the C₄-acetylenes and the butadiene to 1-butene and 2-butene. Hydrogenation can be performed in a conventional manner in a fixed bed or alternately in a catalytic distillation unit. The catalytic hydrogenation unit 11 can employ any suitable hydrogenation catalyst such as, for example, palladium on alumina, in a packed bed. Hydrogen can be added at a level representing 1.0 to 1.5 times the hydrogen required to hydrogenate the dienes and acetylenes to olefins. The conditions are variable depending on reactor design. If, for example, the catalytic hydrogenation unit 11 is operated as a catalytic distillation unit, the temperature and pressure are consistent with fractionation conditions. The C₄ fraction 12 produced by catalytic hydrogenation unit 11 contains mainly 1-butene, 2-butene, isobutene and a small amount of other components such as normal and iso butanes.

Under such conditions of hydrogenation, hydroisomerization reactions also occur. Significant quantities of 2-butene are formed by the hydroisomerization of 1-butene, which is produced by the hydrogenation of butadiene. The fraction 12, now containing only olefins and paraffins, is processed for the removal of the isobutylene fraction in unit 13. There are a number of processes that will accomplish this.

In a preferred process the isobutene is removed by catalytic distillation combining hydroisomerization and superfractionation in unit 13. The hydroisomerization converts 1-butene to 2-butene, and the superfractionation removes the isobutene in stream 14, leaving a relatively pure 2-butene stream 15 containing some isobutane and n-butane. The advantage to converting the 1-butene to 2-butene in this system is that the boiling point of 2-butene (1° C. for the trans isomer, 4° C. for the cis isomer) is further away from the boiling point of isobutylene (−7° C.) than that of 1-butene (−6° C.), thereby rendering the removal of isobutene by superfractionation easier and less costly and avoiding the loss of 1-butene overhead with the isobutylene. The relatively pure 2-butene stream 15 is used as a feed stream F for the olefin isomerization process described below.

Alternately, unit 13 (isobutylene removal) could be an MTBE unit where isobutylene is removed via reaction with methanol to form MTBE. The remaining normal olefins (stream 15) consisting of 1 and 2-butenes, are relatively untouched in this reaction.

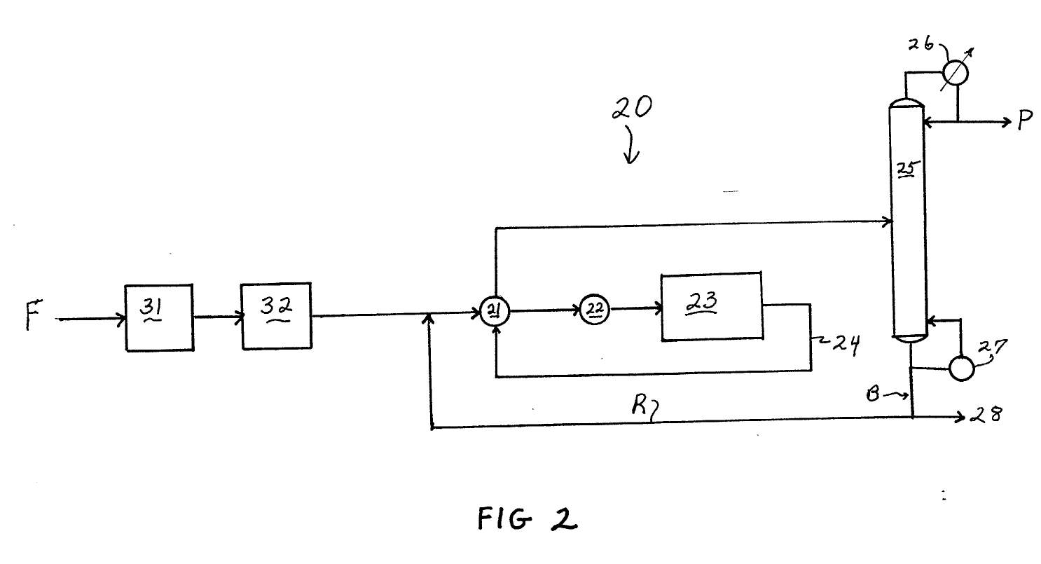

Referring now to FIG. 2, the isomerization of a feed F containing primarily 2-butene by the system 20 is illustrated.

First the feedstock F is passed through guard bed 31 to remove molecular oxygen, and guard bed 32, which is a 13×molecular sieve. Processes of the prior art (e.g., U.S. Pat. No. 4,217,244 to Montgomery) include passing feedstock F through a 13×molecular sieve prior to introduction into the isomerization reactor. A 13×molecular sieve removes polar compounds such as water and alcohols but does not remove molecular oxygen. Surprisingly, we have found that in addition to removal of the polar compounds, removal of trace levels of molecular oxygen down to ≦1 ppmv will improve catalyst life. This is accomplished in guard bed 31 by use of special absorbent beds, most typically including copper in a reduced state on a suitable support. The oxygen reacts with the copper to form copper oxide and the molecular oxygen is thus removed from the olefin-rich feed stream. Oxygen guard bed 31 is preferably located upstream of 13×guard bed 32 since water may be formed within the molecular oxygen removal bed 31. Following the guard beds 31 and 32, deoxygenated feed F is mixed with a 2-butene recycle stream R and is sent to a first heat exchanger 21 wherein heat is recovered from the effluent stream 24 of the isomerization reactor 23. Feed F is then sent to a heater 22 which raises the temperature of the feed stream to a preferred isomerization temperature of from 300° C. to 600° C., preferably 340° C. to 500° C. Feed F then enters isomerization reactor 23 where it is contacted with an isomerization catalyst, such as described below, at the isomerization temperature. Reaction pressure is not critically important and can range from subatmospheric to more than 400 psig. Reactor 23 can be any reactor suitable for isomerization such as axial flow, radial flow or parallel flow. The catalyst can be in the form of particulate such as powder, pellets, extrudates, etc.

As stated above, higher temperatures shift the reaction equilibrium to favor the production of 1-butene. At the isomerization temperatures indicated above, a 2-butene conversion of 20 percent to 30 percent to 1-butene is achievable.

The effluent 24 is passed through heat exchanger 21, for heat recovery and is then sent to a fractionator 25 for separation of the 1-butene and 2-butene isomers. Condenser 26 recycles 1-butene for reflux. A relatively pure 1-butene stream is drawn off as overhead product P. A bottoms fraction B containing unreacted 2-butene and butanes is produced. A portion of the 2-butene rich bottoms is sent via recycle stream R back to the feed F. A small portion of the bottoms fraction is bled off at stream 28. Since the feed F contains some butanes, which are unreacted and are separated with the fractionator bottoms, the butanes would accumulate through recycling, thereby wasting energy if the bottoms were not bled. One skilled in the art would adjust the amount of bottoms bled off stream 28 and recycled via stream R to achieve the most economical operation of the system 20.

Useful isomerization catalysts include basic metal oxides such as magnesium oxide, calcium oxide, barium oxide, and lithium oxide, either individually or in combination. Other oxides such as sodium oxide or potassium oxide can be incorporated into the catalyst as promoters. The preferred catalyst for use in the isomerization process described herein is magnesium oxide (MgO) and the invention will be described in terms of magnesium oxide, although it should be understood that the other basic metal oxides mentioned above are also contemplated as being within the scope of the invention. The magnesium oxide catalyst can be in the form of powder, pellets, extrudates, and the like.

One of the problems associated with magnesium oxide and other basic oxide catalysts is the shortness of the duration of its catalytic activity under favorable isomerization conditions of high temperature to form the alpha olefin. Conventional magnesium oxide (or other basic metal oxide) catalyst experiences a rapid drop of catalyst activity after about 20-40 hours of operation on-stream. The deactivation rates as measured by the loss of conversion of 1-butene to 2-butene are approximately 0.3 percent conversion loss/hr or higher. Such a rapid loss of initial activity either as a fresh catalyst or regenerated catalyst renders the process economically less feasible and inhibits the wider use of magnesium oxide as an isomerization catalyst.

Typically, the catalyst is treated in dry inert gas to remove residual water and carbon dioxide prior to use in the isomerization reaction. Water and carbon dioxide are generally chemically bound to the magnesium oxide in the form of magnesium hydroxide and magnesium carbonate. Although not wishing to be bound by any explanation, it is believed that these compounds act as acid sites which promote the fouling reactions that limit the onstream cycle life of the system.

A preferred catalyst for use in the olefin isomerization process is disclosed and described in U.S. Patent application Serial No. ______ filed concurrently herewith (under Attorney Docket No. 1094-7), which is herein incorporated by reference.

Prior to its initial use in an olefin isomerization reaction the magnesium oxide (or other basic metal oxide catalyst) is heated in a dry inert atmosphere at sufficiently high temperature to remove substantially all activity-affecting amounts of water and carbon dioxide. A suitable activation treatment of the magnesium oxide catalyst can be performed in one or more steps. Preferably, a two step process is employed wherein the magnesium oxide catalyst is preheated for at least about 15 hours at a temperature of least 350° C. in a dry inert atmosphere as a drying first step. More particularly, a flow of dry pure inert gas such as nitrogen is passed through a bed of magnesium oxide catalyst at a temperature of at least about 350° C. for at least about 15 hours while the effluent is monitored for release of water and carbon dioxide. The effluent water concentration is brought down to less than 1 ppm.

In a preferred second step the catalyst is activated by contact with an inert gas (e.g., nitrogen) at about at least 500° C., preferably at about at least 550° C. for at least about 6 hours.

A significant improvement in catalyst life is achieved by removing oxygen which often accompanies nitrogen as an impurity. Deoxygenation can be performed by any conventional process known in the art. Thus, while conventional sources of nitrogen (for example, nitrogen derived from the cryogenic fractionation of air) contain up to 10 ppm or more of oxygen, removal of this oxygen by, for example, passing the nitrogen through an O₂ adsorption bed prior to its use in the catalyst treating process described above, results in a catalyst having a significantly longer life. Preferably, the deoxygenated nitrogen contains no more than about 5 ppm of oxygen, more preferably no more than about 2 ppm of oxygen, and most preferably no more than about 1 ppm of oxygen. Substantially all activity affecting amounts of carbon dioxide and water are removed by using deoxygenated nitrogen.

While the treatment process described above improves the catalyst performance enabling operation of the isomerization for a period of over 150 hours, the olefin isomerization process must be cycled to allow for regeneration of the catalyst to remove coke deposits. The benefit of the dry-out achieved by the treatment process set forth above is lost on the second cycle when standard regeneration procedures are employed.

The regeneration process herein restores the catalyst to substantially its initial fresh condition and includes a decoking step, preferably followed by a high temperature catalyst reactivation step.

The decoking step substantially completely removes all activity affecting amounts of coke, water and carbon dioxide from the catalyst and restores the catalyst to substantially its initial level of activity. The high temperature reactivation step removes substantially any remaining traces of water and/or carbon dioxide capable of affecting catalyst activity for further extension of catalyst life.

More particularly, the decoking step includes contacting the catalyst with a flowing atmosphere containing a dry inert gas (e.g., nitrogen) and an oxidizing agent (e.g., oxygen) at a regeneration temperature of at least about 500° C. for at least about 6 hours, preferably about 12 hours, and most preferably about 18 hours to substantially completely remove all coke from the catalyst. The regeneration proceeds in steps of gradually increasing temperature and oxygen concentration as described in U.S. Pat. No. 4,217,244, which is herein incorporated by reference. Pure, dry air is preferably used as the flowing atmosphere.

Preferably, the decoking step includes preheating the catalyst by contacting the catalyst with a flowing atmosphere of dry inert gas containing at least about 2 percent of oxygen for at least about 6 hours at a temperature of at least about 460° C. prior to contacting the catalyst with the 20 percent oxygen atmosphere at 500° C. for 18 hours, the total decoking time being at least about 24 hours.

The high temperature reactivation step includes contacting the decoked catalyst with a flowing atmosphere of pure, dry inert gas (e.g. nitrogen) for at least about 6 hours at a temperature of at least about 500° C., and preferably about 50° C. higher than the decoking temperature (i.e., at least about 550° C.) to desorb any remaining water and carbon dioxide. The nitrogen is preferably pretreated to remove oxygen as discussed above. The deoxygenated nitrogen preferably contains no more than about 5 ppm oxygen, more preferably no more than about 2 ppm oxygen, and most preferably no more than about 1 ppm oxygen.

Prior to regeneration the catalyst is preferably flushed with dry inert gas at ambient or elevated temperature to remove hydrocarbons or other volatile components.

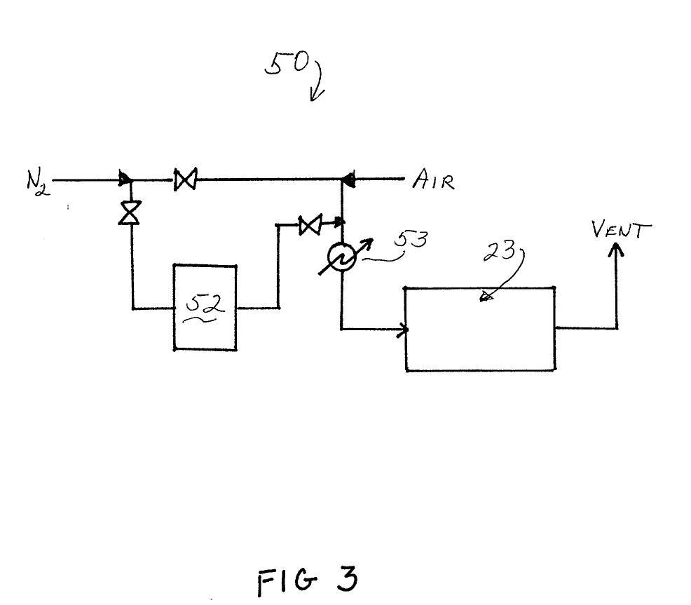

Referring now to FIG. 3, a regeneration/activation system is shown in association with reactor 23. During the regeneration step, a combination of inert gas, i.e., nitrogen, and air are used in progressive steps of increasing oxygen concentration and temperature to remove the coke from the catalyst. The nitrogen is first bypassed around an oxygen removing guard bed 52 and mixed with air. Heat exchanger 53 adjusts the temperature of the gas entering reactor 23 to the desired degree. The effluent gas is vented from the system or sent to heat recovery. There is no need to remove oxygen from the inert gas at this point since oxygen is being used to burn the coke. Following the regeneration, a reactivation process occurs as described above. As the final step in this process, a dry inert gas (nitrogen) is passed over the catalyst at a temperature approximately 50° C. higher than the maximum temperature during the regeneration cycle. This allows for the removal of the water and CO₂ that were chemically bonded to the MgO during regeneration as hydroxides and carbonates. This final inert step uses a deoxygenated gas to prevent any oxygen from physically adsorbing on the catalyst during the final sweep operation. In this step the nitrogen is now passed through the oxygen removing guard bed 52. No air is used in this step. The inert gas, now containing less than about 1 ppm oxygen passes through the heat exchanger 53 where the temperature is adjusted to the desired level. The gas then goes to reactor 23 where it is used in the final reactivation step. The combination of a totally molecular oxygen free bed following regeneration/activation and the continuous removal of any trace molecular oxygen during operation results in long catalyst life during the reaction cycle.

Various aspects of the invention are illustrated by the Example given below:

### EXAMPLE 1

To illustrate the influence of trace amounts of molecular oxygen on the catalyst life, two identical MgO catalyst samples, designated herein as Sample A and Sample B, were subjected to identical initial dryout procedures. They were then used to isomerize 1-butene to 2-butene at elevated temperatures. After some period of operation, both samples lost activity and were regenerated. Both samples were conventional grade magnesium oxide containing 692 ppm iron, 2335 ppm sulfur, 3522 ppm calcium and less than 250 ppm sodium. After a nitrogen flush, both of the coked samples were exposed to nitrogen containing progressively increasing temperatures and molecular oxygen concentrations. The last regeneration step was exposure to nitrogen containing 21 percent molecular oxygen for 18 hours at 500° C. Thereafter, a high temperature reactivation step was performed on all samples by exposing the samples to dry nitrogen at 550° C. However, Sample A, was treated with a purified nitrogen containing no more than 1 ppm of molecular oxygen in accordance with the process of the present invention, the nitrogen being purified by passing it through a molecular oxygen adsorption bed. For comparison, Sample B was treated with nitrogen from a conventional source containing about 10 ppm or more of molecular oxygen.

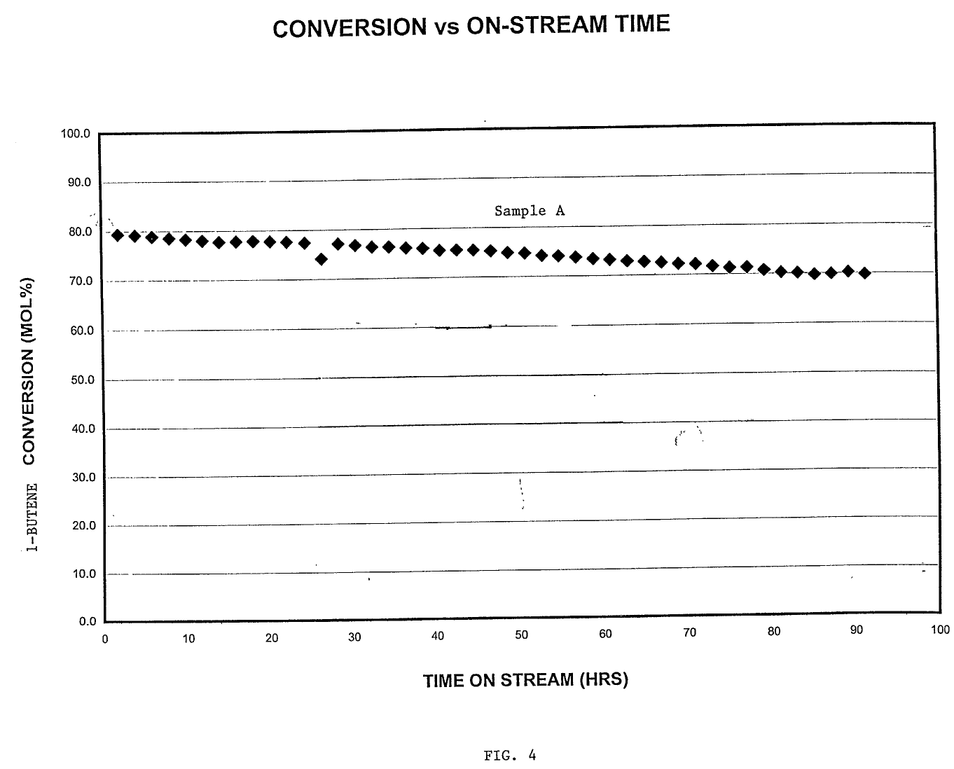

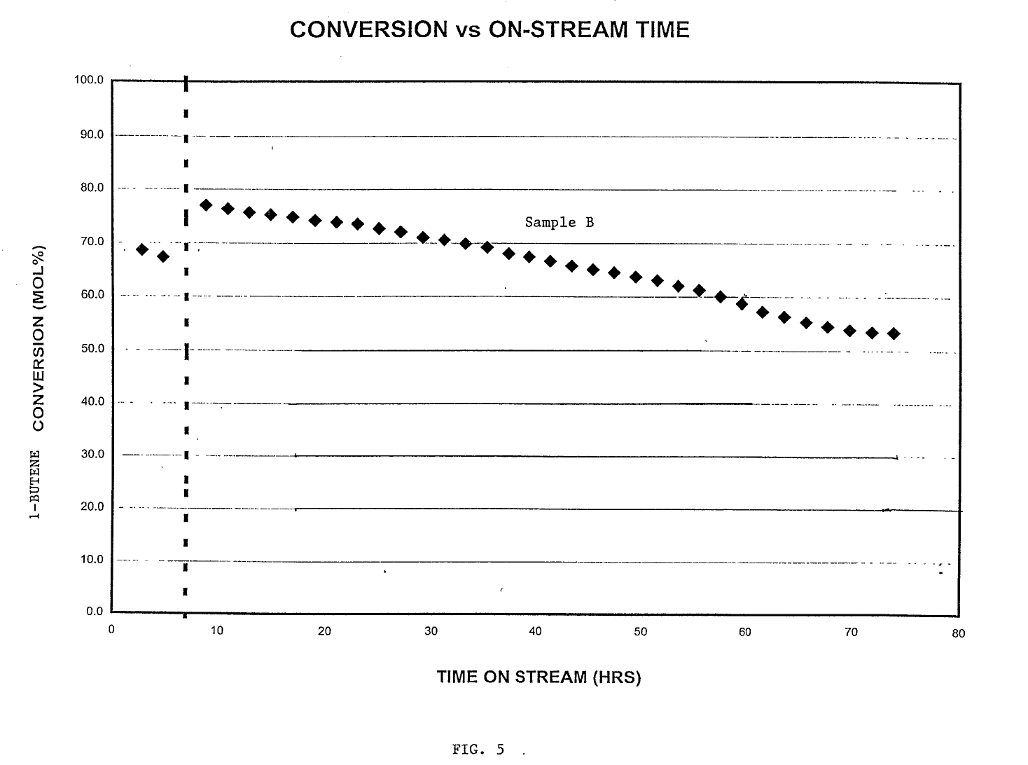

The samples were then individually tested in the isomerization of 1-butene. The 1-butene was passed through an oxygen guard bed. Both samples were tested in an isomerization reaction conducted at approximately 75 psig, 510° F. and 9 WHSV. The feed stream included 65 percent diluent. The conversion of 1-butene to 2-butene in mol % was monitored during the isomerization. The results are set forth below in Table II and graphically illustrated in FIGS. 4 and 5.

TABLE II

| | Sample A | Sample B |
|---|---|---|
| Catalyst | MgO | MgO |
| Initial 1-C₄ | 79.9% | 77% |
| conversion | | |
| (mol %) | | |
| Final 1-C₄ | 69.8%/93.5 hr | 53.5%/65 hr |
| conversion | | |
| (mol %)/hr | | |
| Deactivation rate | 0.108%/hr | 0.37%/hr |
| (% conversion loss/hr) | | |

As can be seen from the above results, the process of the present invention reduced the deactivation rate of the magnesium oxide catalyst to less than one third the deactivation rate of the comparison sample.

It will be understood that various modifications may be made to the embodiments described herein. Therefore, while the above description contains many specifics, these specifics should not be construed as limitations on the scope of the invention, but merely as exemplifications of preferred embodiments thereof. Those skilled in the art will envision many other possible variations that are within the scope and spirit of the invention as defined by the claims appended hereto.

What is claimed is:

1. A process for activating a basic metal oxide isomerization catalyst which comprises at least one step of contacting the basic metal oxide catalyst under activation conditions with a dry inert gas containing not more than about 5 ppm molecular oxygen by volume.

2. The process of claim 1 wherein the inert gas contains no more than about 2 ppm of molecular oxygen.

3. The process of claim 1 wherein the inert gas contains no more than about 1 ppm of molecular oxygen.

4. The process of claim 1 wherein the inert gas is nitrogen.

5. The process of claim 1 wherein the activation conditions of the at least one step include a temperature of at least about 550° C. and a period of time of at least about 6 hours.

6. The process of claim 1 wherein the basic metal oxide is selected from the group consisting of magnesium oxide, calcium oxide, barium oxide, lithium oxide and combinations thereof.

7. The process of claim 1 wherein the basic metal oxide is magnesium oxide.

8. The process claim 1 further including the step of decoking the catalyst prior to contacting the catalyst with dry inert gas, wherein decoking the catalyst comprises contacting the catalyst with an inert gas combined with at least about 2 percent by weight molecular oxygen at a temperature of at least about 460° C. for at least about 6 hours.

9. The process of claim 8 wherein decoking the catalyst further comprises contacting the catalyst with an inert gas combined with at least about 20 percent molecular oxygen at a temperature of at least about 500° C. for at least about 18 hrs.

10. A basic metal oxide catalyst for isomerization treated in accordance with the process of claim 1.

11. The basic metal oxide catalyst of claim 10 wherein the basic metal oxide is selected from the group consisting of magnesium oxide, calcium oxide, barium oxide, lithium oxide and combinations thereof.

12. The basic metal oxide catalyst of claim 11 wherein the basic metal oxide is magnesium oxide.

13. A process for isomerizing an olefinic feedstock comprising:
a) providing a basic metal oxide olefin isomerization catalyst;
b) activating the basic metal oxide olefin isomerization catalyst by contacting the basic metal oxide catalyst under activation conditions with at least one step of using a dry inert gas containing not more than about 5 ppm molecular oxygen by volume;
c) contacting the olefinic feedstock with the activated basic metal oxide catalyst under olefin isomerization conditions to provide an isomerized product.

14. The process of claim 13 wherein the basic metal oxide catalyst is selected from the group consisting of magnesium oxide, calcium oxide, barium oxide, lithium oxide and combinations thereof.

15. The process of claim 13 wherein the basic metal oxide catalyst is magnesium oxide.

16. The process of claim 13 wherein the inert gas contains no more than about 2 ppm of molecular oxygen.

17. The process of claim 13 wherein the inert gas contains no more than about 1 ppm of molecular oxygen.

18. The process of claim 13 wherein the inert gas is nitrogen.

19. The process of claim 13 wherein the process further includes the step of reducing the content of molecular oxygen in the olefinic feedstock prior to contacting the olefinic feedstock with the basic metal oxide catalyst.

20. The process of claim 19 wherein the step of reducing the content of molecular oxygen of the olefinic feedstock comprises contacting the olefinic feedstock with a reduced metal.

21. The process of claim 20 wherein the reduced metal is copper.
